(19) United States
(12) Patent Application Publication
(10) Pub. No.: US 2010/0209516 A1
(43) Pub. Date: Aug. 19, 2010

(54) Triggered Drug Release Via Physiologically Responsive Polymers

(76) Inventors: Jason J. Benkoski, Ellicott City, MD (US); Andrew F. Mason, Silver Spring, MD (US); Lance M. Baird, Baltimore, MD (US); Jennifer L. Sample, Bethesda, MD (US)

Correspondence Address: THE JOHNS HOPKINS UNIVERSITYAPPLIED PHYSICS LABORA;OFFICE OF PATENT COUNSEL, 11100 JOHNS HOPKINS ROAD, MAIL STOP 7-156, LAUREL, MD, 20723-6099, US

(21) Appl. No.: 12/702,483

(22) Filed: Feb. 9, 2010

Related U.S. Application Data
(60) Provisional application No. 61/152,290, filed on Feb. 13, 2009.

(51) Int. Cl. A61K 9/14; A61K 39/395; A61K 31/713; A61K 38/16; A61P 31/00; A61P 29/00; A61P 3/10; A61P 9/00; A61P 7/02; A61P 37/08

(52) U.S. Cl. 424/490; 424/130.1; 514/44 R; 514/12; 977/906

(57) ABSTRACT

A drug delivery system, product and method which effectuates delivery of appropriate amounts of a pharmaceutically active agent only upon stimulus of a physiological agent released during a disease event are described. A polymer that can bind to a specific biological stimulus and respond with a specific response is included. The response may be release of a pharmaceutical agent, an optical signal or a change in physical properties of the polymer. The design of associative polymers that are held together using temporary bonds which will dissolve, break apart or swell in the presence of the specific stimulus are described. One embodiment includes a reversible response to a biological stimulus.

### CROSS REFERENCE TO RELATED APPLICATIONS

This application claims priority to U.S. Provisional Application No. 61/152,290, filed Feb. 13, 2009.

### BACKGROUND

1. Field of the Invention

This invention relates to the field of pharmaceuticals release in response to physiological conditions.

2. Background of the Invention

When a drug is added directly to the bloodstream, the drug delivery profile is typically marked by a sharp increase in concentration to a peak above the optimum therapeutic range. Depending on the dynamics of the particular drug in the body, drug then decreases rapidly in concentration until it falls below the optimum therapeutic range. Exceeding the therapeutic range can be toxic, whereas undershooting the range may produce no therapeutic benefit. The current solution to this problem is “controlled release,” which maintains the concentration within the optimum range for extended periods of time. Even with controlled release technology, certain disorders may not require constant delivery of a pharmaceutical. In fact, overmedication may be highly undesirable in cases where a given drug may have adverse side effects.

More recently, researchers have turned to “targeted drug delivery,” which concentrates the therapeutic at the sire where treatment is needed. An example would be a chemotherapeutic agent that targets only malignant tumor cells, but not healthy non-cancerous cells. Targeting therapeutics locally is an attractive alternative to controlled drug release because it allows one to administer lower overall concentrations of a given drug, which minimizes systemic side effects while ensuring local concentrations within the optimum therapeutic range. Targeted drug delivery is the ideal solution for ailments that are spatially non-uniform, because they concentrate in one local region of the body, but have low or negligible concentrations systemically.

Controlled release systems using hydrogels have been reported. (Yang, H et al., Engineering target-responsive hydrogels based on aptamer-target interactions, J. Am. Chem. Soc. 130(20):6320-6321, 2008; Miyata, T. et al. Preparation of an antigen-sensitive hydrogel using antigen-antibody bindings, Macromolecules, 32(6):2082-2084, 1999; Parmpi, P. and Kofinas, P, Biomimetic glucose recognition using molecularly imprinted polymer hydrogels, Biomaterials, 25(10):1969-1973, 2004; Zhang, R. et al. A smart membrane based on an antigen-responsive hydrogel, Biotechnol. Bioeng. 97:976-984, 2007; Miyata, T. et al., A reversibly antigen-responsive hydrogel, Nature, 399:766-769, 1999; Lu, Z-R. et al., Antigen responsive hydrogels based on polymerizable antibody Fab′ fragement, Macromolec. Biosci. 3(6):296-300, 2003; and Brownlee, M. and Cerami, A., A glucose-controlled insulin-delivery system: semi-synthetic insulin bound to lectin, Science, 206:1190-1191, 1979).

Earlier reports concentrated on swelling of gel backbones directly, by changes in temperature, pH, and in some cases, glucose levels. Swelling of hydrogels in the presence of various biological molecules has been the focus of several groups. A glucose responsive hydrogel by Parmpi et al. used poly(allylamine hydrochloride) and D-glucose 6-phosphate monobarium salts to construct a molecularly imprinted polymer (MIP) hydrogel. Zhang et al. manufactured hydrogels composed of dextran with divinyl sulfone (DVS) as a cross linking agent. Zhang's hydrogel used FITC antigens and sheep anti-FITC antibody to regulate diffusion through the gel. Miyata et al. synthesized polyvinyl acrylamide backbone gels which swelled upon the interruption of a rabbit IgG antigen and a goat anti-rabbit IgG antibody interaction to allow swelling of the acrylamide gel in response to the presence of exogenous rabbit IgG. Miyata's gel was made by incorporating rabbit IgG antibody-vinyl polymers into an acrylamide gel crosslinked with N,N′,N′-tetramethylethylenediamine (TEMED) and in the presence of goat anti-rabbit IgG antibodies.

Dissolution of hydrogels was reported by Yang et al. (WO 2009/146147 A2 (to Tan, et al.); Yang, supra, 2008) who incorporated nucleic acid aptamers onto polyacrylamide backbones gels which dissolved in the presence of adenosine or thrombin. Aptamers have been used in conjunction with nanomaterials for use in detection systems, (Chiu, T-C and Huang, C—C, Aptamer-functionalized nano-bio sensors, Sensors, 9:10356-10388, 2009) as well.

Triggered drug release is also being developed in the art, but these systems involve separate sensors, electronics and active release involving valves or active injection of drug contents; all of which involve costly surgery, costly supplies and medical monitoring.

There exists a need in the art for a drug release system which can be customized to respond at a clinically appropriate level in vivo, to a physiological event with no outside intervention.

### SUMMARY OF THE INVENTION

The present invention is drawn to a drug delivery system, product and method that effectuates delivery of appropriate amounts of a pharmaceutically active agent only upon stimulus of a physiological agent released during a disease event. The invention includes a polymer that can bind to a specific biological stimulus and respond with a specific response. The response may be release of a pharmaceutical agent, initiation of an optical signal or a change in physical properties of the polymer. The invention includes the design of associative polymers that are held together using temporary bonds which will break apart in the presence of the specific stimulus. By breaking these bonds, one can cause a solid polymer to dissolve in water, cause a quenched fluorophore to begin fluorescing, or cause a polymer to expand and swell with water. The third example of the expanding hydrogel includes a reversible response to a biological stimulus.

### BRIEF DESCRIPTION OF THE FIGURES

FIG. 1A illustrates an unbound ligand and antibody.

FIG. 1B shows the antibody/ligand pair bound together and thus cross-linking the backbone of the gel.

FIG. 1C depicts the introduction of a structurally-similar antigen and the displacement of the crosslinked antibody/antigen binding pair as shown in FIG. 1B.

FIG. 2A depicts the dissolution of a nanocomposite hydrogel of the invention upon binding with a target antigen.

FIG. 2B depicts the dissolution of a polymer of the invention upon binding with a target antigen.

FIG. 3A shows chemical formulas of the thromboxane and prostaglandin molecules that could trigger a response in a polymer of the invention (PRIOR ART).

FIG. 3B depicts a biofunctionalized nanoparticle interacting with an extracellular matrix substrate.

FIG. 4 depicts components of nanocomposite hydrogels.

FIG. 5A shows EDC/NHS coupling of antigen.

FIG. 5B shows covalent coupling of NHS (amine)-PEG to antibody.

FIG. 5C shows the final synthesis step in which multi-arm NHS-PEG is mixed with antibody-coated nanoparticles.

FIG. 6 depicts aggregation of microparticles using polymers with varying numbers of polymer arms.

FIG. 7 is a light micrograph of an aggregated nanocomposite hydrogel.

FIG. 8A illustrates the protective gel with large avidity.

FIG. 8B shows the initiation of the cascade leading to the gel breakup.

FIG. 8C demonstrates the continuing cascading dissociation of the gel.

FIG. 8D shows the final dissolution of the nanocomposite gel.

FIG. 9A depicts a SiO₂ complex embodiment of the invention.

FIG. 9B shows a micrograph of a polymer complex using the SiO₂ molecule of FIG. 8A.

FIG. 10 displays surface Plasmon resonance data for multi-arm polymers functionalized with a prostaglandin E2 antibody.

### DETAILED DESCRIPTION OF THE INVENTION

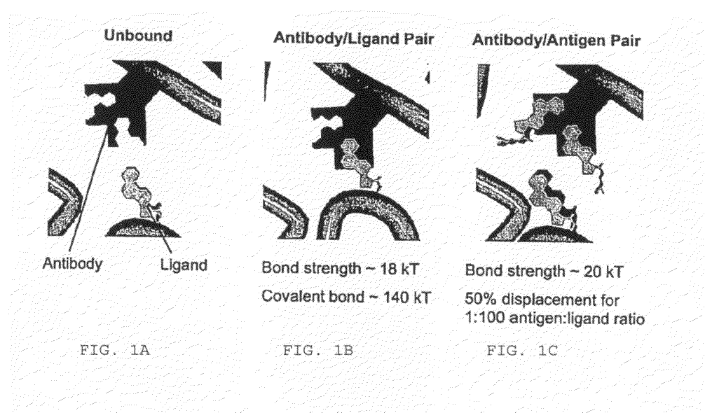

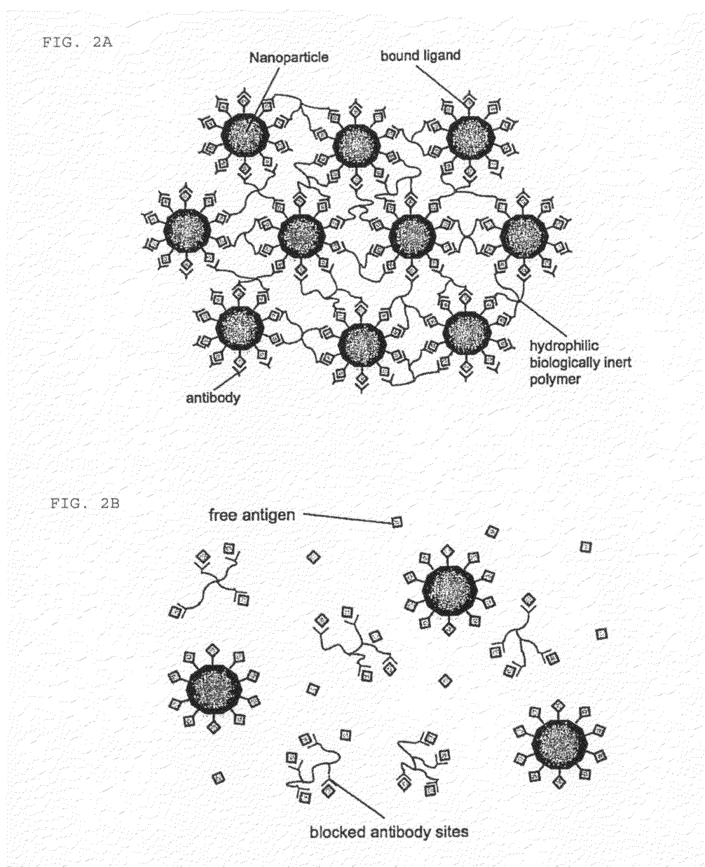

The present invention is drawn to a method, system, and product that allows physiologically triggered drug release. With a goal of the utility of a physiologically triggered drug release, the invention includes a polymer backbone bound to a molecule designed to sense a physiological change, such as an allergic response, and then release an appropriate therapeutic, such as an antihistamine, that is triggered by and in proportion to the instantaneous physiological conditions (FIGS. 1-2). The self-regulated drug delivery system of the invention performs the work of both a sensor and an automated dispenser.

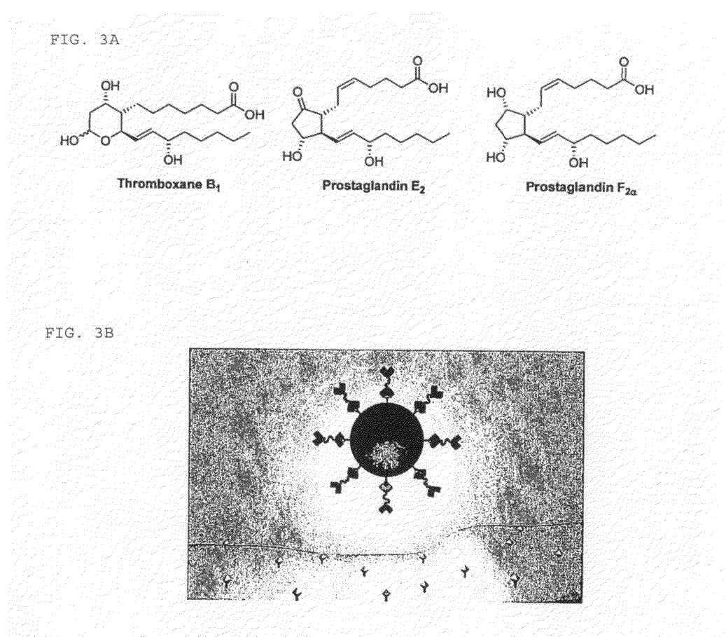

As illustrations of the invention, FIGS. 1A-C illustrate how the attachment of ligands and antibodies to the polymer backbone can lead to associative crosslinks that form a hydrogel network. The Figures further illustrate the difference between an antibody/antigen pair and a bond between the same antibody and a ligand that is structurally related to, but different from, the antigen. FIG. 1A illustrates an unbound ligand and antibody, whereas FIG. 1B shows the antibody/ligand pair bound together and thus linking the backbone of the gel together. FIG. 1C depicts the introduction of an exogenous-supplied antigen and based upon the bond strength, the displacement of the crosslinked antibody/antigen binding pair as shown in FIG. 1B. FIG. 2 depicts the dissolution of the nanocomposite hydrogel of the invention upon binding with a target antigen. FIG. 3A shows chemical formulas of the thromboxane and prostaglandin molecules that could trigger the reaction of FIGS. 2A and 2B. FIG. 3B depicts a biofunctionalized nanoparticle interacting with an extracellular matrix substrate. In addition to releasing therapeutics, possible responses include the generation of an optical signal for standoff detection or the actuation of, for example, a sphincter valve in an implanted device. The binding properties of the molecule to which a physiological molecule will bind and the topological architecture of the macromolecular network are modified to achieve the desired rate of response, specificity and sensitivity.

The invention has medical benefits in that is can simultaneously act as a sensor, dispenser, and therapeutic in a passive, self-regulated package. The product and methods of the invention can remove the need for an expensive implant device, and can reduce the need for continuous medical supervision in some situations. Because the trigger for the polymer of the invention is the physiological production of biomarkers, elevated stress, foods, physical activity, thirst, sweating, an the like, can be used to set off an adverse condition. The response of the invention can be similarly diverse, such as stand-off detection, or the release of depressants, stimulants, irritants, etc.

One embodiment of the invention includes associative antibody/antigen bonds to hold together polymer chains into extended, cross-linked macromolecular assemblies. One aspect of the invention is to replace the antibody/antigen bond with an antibody/ligand bond in which the ligand is different, but structurally related to the antigen, or a molecularly-imprinted polymer (MIPS)/antigen bond. The molecules of the invention exploit the fact that the structurally related ligands bind poorly with antibodies, the same holding true for MIPS/antigen binding. What has been considered a weakness in the art, especially for MIPS binding, is used as an asset in the present invention. The lower binding strength of the MIPS/antigen binding improves the responsiveness of the molecules of the invention to the local environment. The true target molecule will out-compete the engineered ligands or MIPS to effectively break the bonds and dissolve the polymer and thus release the therapeutic agent. In contrast, the glucose responsive MIPs gel reported by Parmpi et al. (Biomaterials, 25(10):1969-1973, 2004) uses the crosslinking agent epichlorohydrin (EPI), and thus the gel would not dissolve upon contact with the target molecule.

Yang et al. (J. Am. Chem. Soc., 130(20):6320-6321, 2008) constructed adenosine- and thrombin-responsive hydrogel linear backbones made from polyacrylamide. Oligonucleotide aptamers were employed to cross-link the chains. Release of encapsulated gold particles was demonstrated in the presence of either thrombin or adenosine, respectively, indicating dissolution of the gel. The response was all or nothing and in contrast to the present invention, no modification of the aptamers was practiced or suggested to allow for graded response or tailoring to elevated concentrations of biological agents that are present in physiological concentrations. There was no discussion in Yang of modifying the strength of the bonding to customize the response or to allow for target concentrations. Furthermore, the target molecule was limited to nucleotides such as DNA or RNA. The present invention applies to essentially all biomolecules.

In contrast to the present inventions, the hydrogel by Parmpi et al. (MIPs and poly(allylamine hydrochloride and GPS-Ba) cross links the get using epichlorohydrin, the hydrogel by Zhang et al. (hydrogels composed of dextran with divinyl sulfone as a cross linking agent), and Miyata et al. (polyvinyl acrylamide backbone gels rabbit IgG antibody-vinyl polymers incorporated into an acrylamide gel crosslinked with N,N,N′,N′-tetramethylethylenediamine (TEMED) and in the presence of goat anti-rabbit IgG antibodies) are all gels crosslinked gels to which the binding groups merely allow swelling of the gel in response to the target agent. The present invention utilizes crosslinks composed of a receptor and ligand which forms weaker bonds than would form with the natural substrate, hence allowing more rapid dissolution of the gel upon contact with the natural target and at even lower concentrations. The present invention also allows the design of the specific binding pairs to modulate the strength and rapidity of the reaction by modulating the number of binding pairs, affinity and/or the avidity of the binding and the introduction of differing binding pairs within one gel to further aid in specificity and timing of dissolution of the gel.

Sensitivity improvements of the invention include the synthesis of increasingly sophisticated polymer architectures with cascading bond breakage that improve sensitivity of the molecule to respond to physiological agents. In one embodiment, multiple antibodies, ligands, nucleic acid or protein aptamers, MIPS or a combination thereof are used in the same assembly. While a single molecule may not unambiguously identify a particular physiological process, two or more molecules may present simultaneously may increase sensitivity and decrease cross-reactivity or improper response (false positives).

Also included in the invention is a feedback method wherein molecules for sensing drug levels and/or physiological target molecules can then shut off the delivery of the drug by binding to ligands on the molecule of the invention and thus causing a conformational change in the polymer which then prevents further release of the therapeutic agent.

“Physiological agents” are molecules which produce a physiological effect on the body of an animal. Internal physiological agents are released within the body of an animal, which produce physiological effects within the animal. Examples would be insulin or prostaglandins. External physiological agents are introduced from outside the body of an animal, but which produce physiological effects within the animal. Examples would include allergens, therapeutic agents or toxins.

“Target molecules” of the invention include physiological agents including, but not limited to, histamines, cortisol, clotting cascade agents such as serotonin, platelet-activating factor (PAF), von Willebrand factor (vWB), Factor VIII, platelet factor 4, and thromboxane A₂ (TXA₂); eicosanoids such as prostaglandin, thromboxane, and prostacyclin; hormones such as dopamine, aldosterone, calcitonin, testosterone, estrogen, insulin, melatonin, thymosin, and calcitriol; erythropoietin (EPO) and thrombopoietin (THPO); antiplasminogen factors; and endotoxin, plasminogen activating factor, tissue factor; immune factors such as, lymphokines, interleukins; and cytokines such as interleukin 1 (IL1), IL2, Th1 and Th2 factors; tumor necrosis factor (TNFα and TNFβ); interfereons (IFN) such as type 1 (IFN-γ, TGF-β, etc.), and type 2 (IL-4, IL-10, IL-13), Il-1α, Il-1β and the like; transforming growth factors (TGF) such as, TGF-β1, TGF-β2 and TGF-β3; chemokines such as, C—C chemokines (RANTES, MCP-1, MIP-1α, and MIP-1b), C—X—C chemokines (IL-8), C chemokines (lymphotactin), and CXXXC chemokines (fractalkine); immunoglobulins, such as IgE, IgG₁; GM-CSF; signal-transducing γ chain for the IL-2R subfamily of receptors (such as IL-4, IL-7, IL-9, and IL-15), such as IL-2R β chain, monomeric IL-2R, dimeric IL-2R βγ, and trimeric IL-2R αβγ, high affinity IL-2R a chain (Tac); Fas; macrophage migration inhibitory factor (MIF) and inducible phosphofructokinase-2 (iPFK-2). The invention also includes glucose regulating molecules, including, catabolic hormones, such as glucagon, growth hormone, cortisol and catecholamines and the anabolic hormone, insulin, as well as glucose levels.

Therapeutic agents include, but are not limited to: anticoagulants such as dipyridamole, warfarin, heparins, including but not limited to enoxaparin (Lovenox), dalteparin (Fragmin), tinzaparin (Innohep), nadroparin (Fraxiparine), reviparin (CLIVARIN) and certoparin (Sandoparin); heparin antidotes, such as hirudin, lepirudin (REFLUDAN), Danaparoid, and bivalirudin (HIRULOG) as well as argatroban (NOVASTAN); adrenaline; glycoprotein (GP) IIB/IIIA; thrombolytic agents such as streptokinase, urokinase-type plasminogen activator (UPA) and tissue-type plasminogen activator (TPA); antiplatelet agents such as ticlopidine (TICLID) and clopidogrel (PLAVIX); COX inhibitors; NSAIDS, including ibuprofen and aspirin. Diabetes therapeutics, such as the alpha-glucosidase inhibitors GLYSET and PRECOSE, biguanides, such as metformins, (GLUCOPHAGE, GLUCOPHAGE XR, and RIOMET), the D-phenylalnin derivative, STARLIX, diphosphophosphatase-4 inhibitor, JANUVIA, meglitinide (PRANDIN), sulfonylureas glimepiride (AMARYL), glyburide (DIABETA), chlorpropamide DIABINESE, glipizide (GLUCOTROL and GLUCOTROL XL), glyburide (GLYNASE and MICRONASE), tolazamide, and tolbutamide, thiazolidinediones ACTOS and AVANDIA, and combination medications, metformin and pioglitazone (ACTOPLUS MET), rosiglitazone and metformin (AVANDAMET), rosiglitazone and glimepiride (AVANDARYL), pioglitazone and glimepiride (DUETACT), glyburide and metformin (GLUCOVANCE), sitagliptin and metformin (JANUMET), and metformin and pioglitazone (METAGLIP); amylin mimetic, pramlintide acetate (SYMLIN), and incretin mimetic, exenatide (BYETTA).

“Nucleic acid” molecules, as used herein include DNA, RNA, polynucleotides and oligonucleotides; synthetic or naturally-occurring. The nucleic acid molecules include any single-stranded sequence of nucleotide units connected by phosphodiester linkages, or any double-stranded sequences comprising two such complementary single-stranded sequences held together by hydrogen bonds. Unless otherwise indicated, each nucleic acid sequence set forth herein is presented as a sequence of deoxyribonucleotides (abbreviated A, G, C and T). However, the term “nucleic acid” includes a DNA molecule or polynucleotide, a sequence of deoxyribonucleotides, or an RNA molecule or polyribonucleotide. The corresponding sequence of ribonucleotides includes the bases A, G, C and U, where each thymidine deoxyribonucleotide (T) in the specified deoxyribonucleotide sequence is replaced by the ribonucleotide uridine (U).

Nucleic acids may originate in viral, bacterial, archobacterial, cyanobacterial, protozoan, eukaryotic, and/or prokaryotic sources. All DNA provided herein are understood to include complementary strands unless otherwise noted. It is understood that an oligonucleotide may be selected from either strand of the genomic or cDNA sequences. Furthermore, RNA equivalents can be prepared by substituting uracil for thymine, and are included in the scope of this definition, along with RNA copies of the DNA sequences isolated from cells or from virion particles. The oligonucleotide of the invention can be modified by the addition of peptides, labels, and other chemical moieties and are understood to be included in the scope of this definition.

Nucleic acid molecules detected or used by the methods or systems of the invention may also include synthetic bases or analogs, including but not limited to fluoropyrimidines, pyrimidines and purine nucleoside analogues include, fluoropyrimidines, including 5-FU (5-fluorouracil), fluorodeoxyuridine, ftorafur, 5′-deoxyfluorouridine, UFT, carboranyl thymidine analogues, FMAUMP (1-(2-deoxy-2-fluoro-D-arabinofuranosyl)-5-methyluracil-5′-monophosphate) and S-1 capecitabine; pyrimidine nucleosides, include deoxycytidine, cytosine arabinoside, cytarabine, azacitidine, 5-azacytosine, gencitabine, and 5 azacytosine-arabinoside; purine analogs include 6-mercaptopurine, thioguanine, azathioprine, allopurinol, cladribine, fludarabine, pentostatin, 2-chloroadenosine, AZT, acyclovir, pencilcovir, famcyclovir, didehydrodideoxythymidine, dideoxycytidine, -SddC, ganciclovir, dideoxyinosine, and/or 6-thioguanosine, for example, or combinations thereof.

“Proteins” as used herein include peptides and polypeptides. A protein is a large molecule composed of one or more chains of nitrogen-containing amino acids linked together in a peptide linkage, in a specific order determined by the base sequence of nucleotides in the DNA coding for the protein. Examples of proteins include whole classes of important molecules, among them enzymes, hormones, antibodies and toxins. Proteins as used herein are composed of 20 standard amino acids, or may contain synthetic or naturally occurring non-standard amino acids. The amino acids present in the protein may be aromatic, D or L configuration, modified or having an R or S chirality. Such proteins may contain amino acids with posttranslational modifications. Such posttranslational modifications include but are not limited to carboxylation of glutamate, hydroxylation of proline or the addition of long hydrophobic groups can cause a protein to bind to a phospholipid membrane. Proteins may originate in viral, bacterial, archobacterial, cyanobacterial, protozoan, eukaryotic, and/or prokaryotic sources.

The proteins used by the methods or systems of the invention include polypeptides and/or peptides and further include proteins that are part of a chimeric or fusion protein. Said chimeric proteins may be derived from species which include primates, including simian and human; rodentia, including rat and mouse; feline; bovine; ovine; including goat and sheep; canine; or porcine. Fusion proteins may include synthetic peptide sequences, bifunctional antibodies, peptides linked with proteins from the above species, or with linker peptides. Polypeptides of the invention may be further linked with detectable labels, metal compounds, cofactors, chromatography separation tags or linkers, blood stabilization moieties such as transferrin, or the like, therapeutic agents, and so forth. The proteins/peptides of the invention may originate in viral, bacterial, archobacterial, cyanobacterial, protozoan, eukaryotic, and/or prokaryotic sources.

Antibodies used by the methods or systems of the invention include an antibody which is labeled with a labeling agent selected from the group consisting of an enzyme, fluorescent substance, chemiluminescent substance, horseradish peroxidase, alkaline phosphatase, biotin, avidin, electron dense substance, and radioisotope. The antibody of this invention may be a polyclonal antibody, a monoclonal antibody or said antibody may be chimeric or bifunctional, or part of a fusion protein. The invention further includes a portion of any antibody of this invention, including single chain, light chain, heavy chain, CDR, F(ab′)₂, Fab, Fab′, Fv, sFv, dsFv and dAb, or any combinations thereof.

One embodiment of the invention includes a nanocomposite hydrogel which senses a physiological change, such as an allergic response, and then releases a dose of a therapeutic, that is triggered by and in proportion to the instantaneous physiological conditions. The nanocomposite hydrogel will be made by reverse template synthesis to form well defined nanocomposite hydrogels. From this material, the nanocomposite hydrogel is dissolved when exposed to a specific biomolecule, releasing a drug or other therapeutic (FIGS. 1 and 2).

One example of the use of the invention is in thrombolytic drug delivery (FIGS. 3A and 3B). Treatment approaches include, the prevention of clot formation, drug release during clot formation, and/or the destruction of a clot after thrombosis. The responsive gels can be triggered by specific biochemical markers by temporal on-demand release. Eicosanoids as triggers for the response, as can signaling molecules for inflammation and immune responses. The therapeutic agents would be readily available and the molecules designed for the signaling of the response have suitable chemical functionality.

The scientific underpinnings for this technology relate to associative bonds which are described by an affinity constant, Kₐ, which determines the ratio of free and bound antibody and antigen. If [H] represents the molar concentration of antigen, [R] represents the molar concentration of antibody, and [HR] represents the molar concentration of bound pairs, then:

$${K}_{a} = \frac{[ HR ]}{[ H ] [ R ]} = {e}^{- \frac{{Δμ}_{a}}{{k}_{B} T}}$$

where Δμₐ is the binding energy. Observe how the ratio of bound to free molecules is fixed, and further note how large values of Kₐ correspond to a high percentage of bound pairs.

Typical values of Kₐ for antibody/antigen pairs hover around 10⁹ mol⁻¹, which means that there are essentially no free molecules at equilibrium if stoichiometry is observed. This affinity constant corresponds to a binding energy of about −20 k_{B}T, which is considered a strong bond. Compare this value to the strength of a covalent bond, which is about 140 k_{B}T. A covalent bond is considered to be a very strong bond. A weak bond would have a binding energy of about 1 k_{B}T, which means that it can be easily broken by thermal energy at room temperature. Antibodies can also bind to structurally related molecules. However, the binding strengths with these ligands are weaker. The strength can range between 1 and 20 k_{B}T, depending on how dissimilar the ligand is with the antigen.

Kₐ also holds information about the binding rate constant (kₐ), the dissociation rate constant (k_{d}).

$${K}_{a} = \frac{{k}_{a}}{{k}_{d}}$$

The characteristic residence time (τ) of the bond is further given by the inverse of k_{d}.

$$τ = 1 / {k}_{d}$$

The residence time, or typical lifetime, of the antibody/antigen complex can range from 30 minutes to hours. The relationship between τ and k_{d} is true because of the following equations (which assume that both antibody and antigen diffuse irreversibly away from the source).

$$\frac{\partial [ HR ]}{\partial t} = - {k}_{off} [ HR ] ⇒ [ HR ] = {[ HR ]}_{t = 0} {e}^{- {k}_{d} t}$$

When multiple associative bonds simultaneously act to bind two macromolecules together, the avidity constant, Kₐᵥ, is merely equal to Kₐ^{N}, where N is the number of bonds. Whereas the affinity constants are multiplicative, the binding energies are additive, to give the avidity energy Δμₐᵥ=NΔμₐ.

More generally, we can describe the avidity constants and avidity rate constants in terms of many bonds, even including different types of bonds:

$${K}_{av} = \frac{{k}_{avon}}{{k}_{avoff}} = {\prod }_{i}^{N} \frac{{k}_{ion}}{{k}_{ioff}} = {e}^{{- \sum }_{ i}^{ N} {Δμ}_{i} / {k}_{B} T}$$

which also gives:

$${k}_{avon} = {\prod }_{i}^{N} {k}_{ai}$$

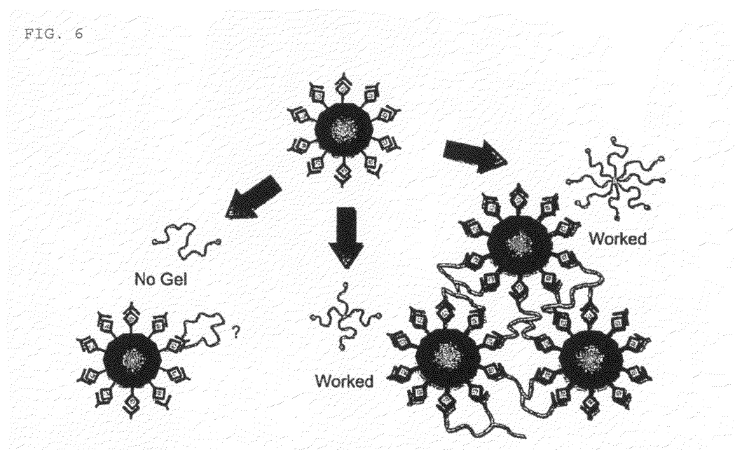

An example of this phenomenon is given in FIG. 6, which displays surface plasmon resonance (SPR) data for a series of multiarm polymers that are end functionalized with a Prostaglandin E2 (PGE2) antibody. The number of antibodies, N, is equal to the number of arms on the branched polymer chains. When exposed to a gold SPR chip that is decorated with a monolayer of PGE2, we see an increase in kₐᵥₒₙ with increasing number of antibodies. Note the observed power law relationship, which agrees with the prediction given above.

Also derived from this relationship is the dissociation rate constant for a molecule with multiple binding sites:

$${k}_{avoff} = {\prod }_{i}^{N} {k}_{di} \\ [ HRHRHR … ] = {[ HRHRHR … ]}_{t = 0} {e}^{- {k}_{dav} t}$$

$${τ}_{av} = {\prod }_{i}^{N} {τ}_{i}$$

The antibody or antigen can be substituted with either a MIPS or structurally related biomolecule (ligand). These changes will decrease Δμₐ and τ. The key to our design will then be the manipulation of the polymer topology so that the affinity residence time (τ) is low, and that avidity energy (Δμₐᵥ) is high. Further manipulation of the topology will be required to enhance the effect of antigen exposure at low concentrations.

### Exploiting Avidity to Design a Bioresponsive Hydrogel

The ability to calculate Kₐᵥ, kₐᵥₒₙ, k_{avoff}, and τₐᵥ is crucial for designing a hydrogel that dissolves in response to a desired concentration of antigen. The key is the use of the Carothers equation:

$${p}_{G} = \frac{2}{{f}_{avg}}$$

where p_{G} is the critical extent of polymerization, and f_{avg} is the average number of reactive sites per molecule. The extent of polymerization (p) is equal to the fraction of reactive sites that have been used in the reaction. It is equal to 0 for pure monomer, and it is equal to 1 after every functional group has reacted.

The Carothers equation predicts the onset of gelation, where gelation is defined as the point where the polymer network begins to act as a solid. In other words, a polymer gel has a finite elastic modulus and does not flow, whereas before gelation, the polymer undergoes viscous flow. f_{avg} is the average number of reactive sites per monomer. If all monomers link end to end, f_{avg}=2 and yields a linear polymer chain. The chain does not technically form a solid until every monomer reacts to form a single polymer chain. This example illustrates the need to include monomers with 3 or more reactive sites in order to form a three-dimensional network that is required to form a gel.

Conventional polymer chains are static in the sense that covalent bonds are permanent. Associative polymers, such as those described in this invention, are decidedly more dynamic. Rather than being permanent, each bond has a characteristic lifetime, τᵢ, which can be thought of as the typical amount of time that the receptor/ligand pair stays together. A typical lifetime for an antibody/antigen pair ranges from 30-60 minutes.

For an associative polymer, the Carothers equation takes on new meaning. p is still the fraction of binding sites that are occupied. However, it becomes more appropriate to think of it as an average value for a given time. Each receptor/ligand pair breaks and reforms many times during observation, but, on average, maybe 80% of the binding sites are occupied. For this situation, p=0.8 and the associative polymer essentially behaves the same way as a covalently bonded polymer with the same p.

The ability to compensate for weaker antibody/ligand bonds is critical to this invention since antibody/antigen bonds are in some reactions too strong. Antibody/antigen bonds have a characteristic lifetime between 30-60 minutes, whereas an antibody/ligand bond can have a lifetime in the range of 3-6 minutes, or less. Shorter lived bonds are more dynamic, and therefore make it possible to design a nanocomposite hydrogel with a faster response time. In summary, the avidity of the nanocomposite hydrogel network is effectively,

Kₐᵥ=Kₐ^{f}^{_{avg}}.

Kₐᵥ can decrease by substituting the antigen with a structurally related, but different, ligand that has a lower Kₐ. Kₐᵥ can be increased by increasing N, the number of antibodies or ligands per monomer. Using these two parameters, a polymer can be made with any desired Kₐᵥ, as needed for the particular therapeutic regimen. Many weak bonds can form a nanocomposite hydrogel that is roughly equivalent to one with few strong bonds. The advantage to using many weak bonds is that weaker bonds break and reform over shorter time scales than stronger bonds. The faster dynamics make it possible for the nanocomposite hydrogel to respond more quickly to a biological stimulus, such as free antigen in solution.

### Technical Requirements

The physiologically responsive polymer of the invention should only dissolve in the presence of a particular biomolecule. In other words, its response must be specific to a single biomolecule. Even weak responses to related molecules may be unacceptable for some applications.

The polymer must also respond rapidly to physiological trigger. For example, if the polymer takes 30 minutes to release an anticlotting agent, the patient may die of a heart attack or stroke before the drug is ever released. The polymer must therefore undergo its change at the instant that it comes into contact with the trigger molecule.

The polymer must dissolve despite low concentration of biomolecule. In most cases, the trigger molecule will be present in micromolar concentrations or less. The design optionally includes an amplification method or chain reaction to generate a significant response to trace amounts of a given trigger.

A technical challenge for this technology is that a given biomolecule may not be unique to disorder. Many proteins and hormones are involved in multiple physiological processes. Their role depends heavily on context: location in the body, concentration, timing, presence of helper molecules, etc. For many disorders, one cannot pinpoint a single molecule that can unambiguously identify the physiological process that is occurring.

The underlying concept behind this technology is the use of associative antibody/antigen bonds to hold together polymer chains into extended, crosslinked macromolecular assemblies. The key insight that makes this technology viable for triggered drug delivery is the replacement of the antibody/antigen bond with an antibody/structurally related biomolecule bond or a MIPS/antigen bond. The invention takes advantage of the relatively weak binding of structurally related biomolecules with antibodies, the same being true for MIPS and antigens. The lower binding strengths of MIPS and antigens improve responsiveness to the local environment. The true target molecule will out-compete the structurally related biomolecules to effectively break bonds and dissolve the polymer. In contrast, the polymer by Lu et al. (Macromolec. Biosci. 3(6):296-300, 2003) is cross-linked using Fab′ fragments which are co-polymerized with a N,N′-methylenebisacrylamide (MBAAm) crosslinker. The antigen-responsive hydrogel of Miyata et al. (Macromolecules, 32(6):2082-2084, 1999) grafted antigen and antibody onto an acrylamide gel crosslinked using N,N,N′,N′-tetramethylethylenediamine (TEMED). The antigen-responsive gels by Zhang et al. (Macromolecules, 32(6):2082-2084, 1999) are cross-linked using divinyl sulfone (DVS) groups. The MIP gel of Parmpi et al. (Biomaterials, 25(10):1969-1973, 2004) are crosslinked using epichlorohydrin (EPI). All of these gels respond to antigen by swelling, not by dissolving, as the gels of the present invention.

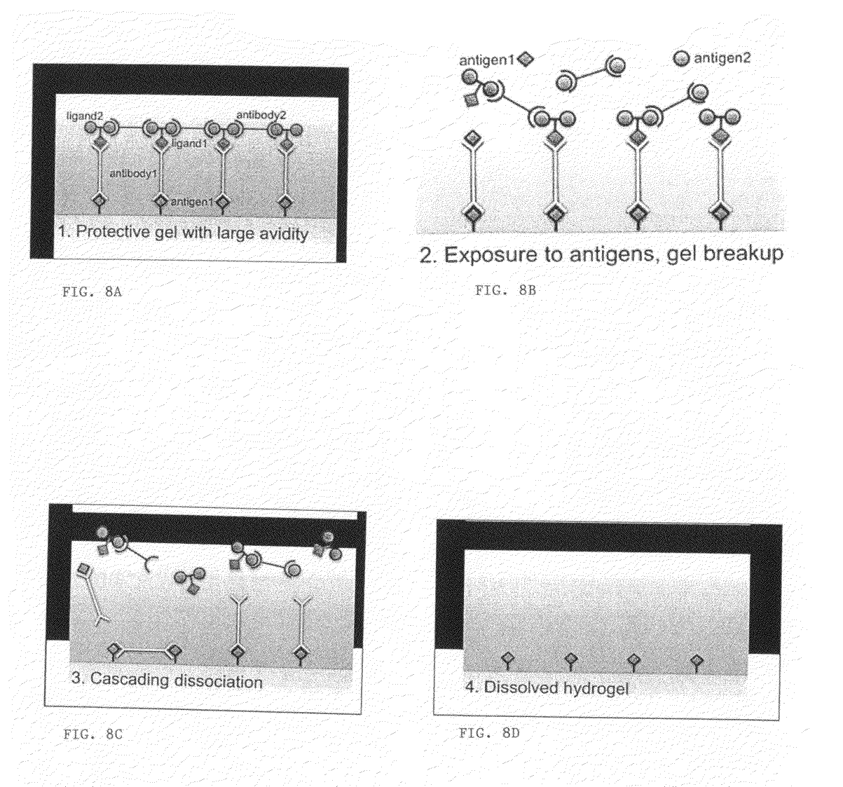

Additionally, synthesis of increasingly sophisticated polymer architectures with cascading bond breakage improves sensitivity (FIG. 8). For example, the polymer is designed so that when a single antibody/structurally related biomolecule bond is broken, it releases a nanoparticle that is coated with hundreds of functional groups that will attack reversible bonds throughout the macromolecular assembly.

To address specificity, multiple and distinct antibodies are used in the same assembly. While a single molecule may not unambiguously identify a particular physiological process, two molecules present simultaneously may ultimately do so.

Shelf life can be an issue when natural antibodies are used. Some antibodies denature over relatively short periods of time; and must be stored at lower temperatures to preserve their bioactivity. Such limitations limit the use of natural antibodies in some cases. Shelf lives of antibody-functionalized polymers are increased by the use of single chains, derivatized antibodies, DNA, oligo-peptides, or MIPS.

The dosage administered depends upon the age, health and weight of the subject, type of previous or concurrent treatment, if any, frequency of treatment, and the nature of the effect desired. The compositions of the invention can be administered by any means that achieve their intended purposes. Amounts and regimens for the administration of the composition according to the present invention can be determined readily by those with ordinary skill in the art. Administration of the composition of the present invention can also optionally be included with previous, concurrent, subsequent or adjunctive therapy in a clinical setting or as part of a dietary regimen.

In addition to the active compounds, a composition of the present invention can also contain suitable carriers acceptable for dietary use and/or pharmaceutical use comprising excipients and auxiliaries which facilitate processing of the active compounds into preparations which can be used pharmaceutically or as a dietary supplement. Suitable formulations for oral administration include hard or soft gelatin capsules, dragees, pills, tablets, including coated tablets, elixirs, suspensions, syrups or inhalations and controlled release forms thereof. Preferably, the preparations, particularly those preparations which can be administered orally and which can be used for the preferred type of administration, such as tablets; dragees, and capsules; softgels; blisters; functional foods, such as power bars, gums, candies, and the like; and functional drinks, such as soft drinks, juices, milks, soy drinks, power drinks, and the like. Drinks such as tea, herbal preparations, coffees and the like are also included in the invention.

Suitable excipients are, for example, fillers such as saccharide, lactose or sucrose, dextrose, sucralose (SPLENDA), aspartame, saccharine, mannitol or sorbitol; cellulose preparations and/or calcium phosphates, such as tricalcium phosphate or calcium hydrogen phosphate; as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone, and may also include preparations comprising natural honey or derivatives. If desired, disintegrating agents can be added such as the above-mentioned starches and also carboxymethyl starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions can be used, which can optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl cellulose phthalate are used. Dyestuffs or pigments can be added to the tablets or dragee coatings, for example, for identification or in order to characterize combinations of active compound doses.

Other preparations which can be used orally include push-fit capsules made of gelatin, as well as soft, sealed capsules made of gelatin and a plasticizer such as glycerol or sorbitol. The push-fit capsules can contain the active compounds in the form of granules which can be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils or liquid paraffin In addition, stabilizers can be added.

Solid dosage forms in addition to those formulated for oral administration include rectal suppositories. The composition of the present invention can also be administered in the form of an implant when compounded with a biodegradable slow-release carrier. Suitable injectable solutions include intravenous subcutaneous and intramuscular injectable solutions. Alternatively, the composition of the invention may be administered in the form of an infusion solution or as a nasal inhalation or spray. Alternatively, the composition of the present invention can be formulated as a transdermal (U.S. Pat. Nos. 5,910,306; 7,037,499; 7,378,097; 7,527,802) or transmucosal patch for continuous release of the active ingredient.

Possible preparations that can be used rectally include, for example, suppositories that consist of a combination of the active compounds with a suppository base. Suitable suppository bases are, for example, natural or synthetic triglycerides, or paraffin hydrocarbons. In addition, it is also possible to use gelatin rectal capsules that consist of a combination of the active compounds with a base. Possible base materials include, for example, liquid triglycerides, polyethylene glycols, or paraffin hydrocarbons.

A formulation for systemic administration according to the invention can be formulated for enteral, parenteral or topical administration. Indeed, all three types of formulation can be used simultaneously to achieve systemic administration of the active ingredient.

Suitable formulations for parenteral administration include aqueous solutions of the active compounds in water-soluble form, for example, water-soluble salts. In addition, suspensions of the active compounds as appropriate oily injection suspensions can be administered. Suitable liphophilic solvents or vehicles include fatty oils, such as sesame oil, or synthetic fatty acid esters, such as ethyl oleate or triglycerides. Aqueous injection suspensions that can contain substances that increase the viscosity of the suspension include, for example, sodium carboxymethyl cellulose, sorbitol, and/or dextran. Optionally, the suspension can also contain stabilizers. Suitable formulations for topical administration include creams, gels, jellies, mucilages, pastes and ointments. The invention provides administratively convenient formulations of the compositions including dosage units incorporated into a variety of containers.

Convenient unit dosage containers include metered sprays, measured liquid containers, measured powdered containers and the like. The compositions can be combined and used in combination with other therapeutic or prophylactic agents. For example, the compounds may be advantageously used in conjunction with other antioxidants, free radical scavengers, and mixtures thereof, or other mixtures as known in the art, (e.g. Goodman & Gilman, The Pharmacological Basis of Therapeutics, 9^{th} Ed., 1996, McGraw-Hill). In another embodiment, the invention provides the subject compounds in the form of one or more pro-drugs, which can be metabolically converted to the subject compounds by the recipient host. A wide variety of pro-drug formulations are known in the art.

Compositions of the present invention are manufactured in a manner which is itself known, for example, by means of conventional mixing, granulating, dragee-making, dissolving, or lyophilizing processes. Thus, preparations for oral use can be obtained by combining the active compounds with solid excipients, optionally grinding the resulting mixture, and processing the mixture of granules, after adding suitable auxiliaries, if desired or necessary, to obtain tablets or dragee cores.

### Working Examples

### Synthesis and Components of Nanocomposite Hydrogel

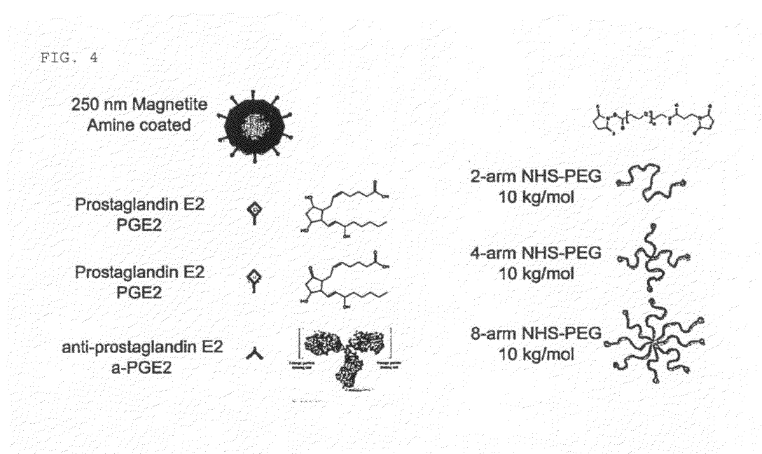

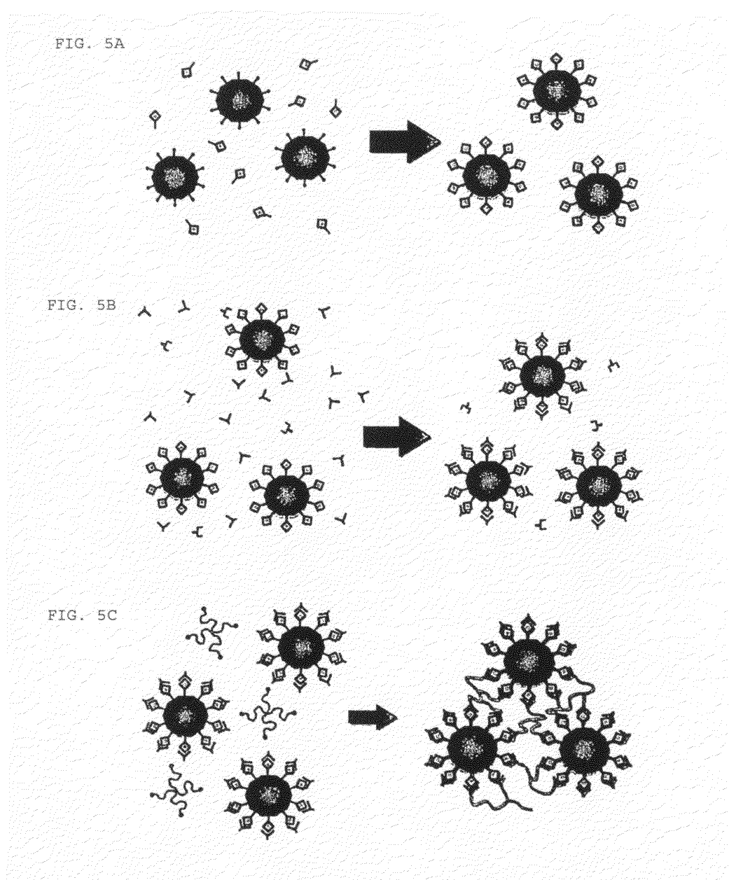

FIG. 4 shows the formation of a nanoparticle hydrogel using backbones of varying length. FIG. 4 depicts hydrogels made with 250 nm magnetite amine-coated nanoparticles, PGE2 antigen and anti-PGE2 antibodies and polymerized with polymer backbones with varying number of branch chains. There are currently three main steps to the synthesis. The first step was to covalently couple the antigen or ligand to the outer surface of a nanoparticle (FIG. 5A). The second step was to noncovalently bind the antibody to the antigen or ligand (FIG. 5B). The third step was to covalently couple an (amine)-NHS end-functionalized polymer to the antibodies, forming a nanocomposite hydrogel (FIG. 5C). FIG. 5A shows EDC/NHS coupling of antigen to nanoparticles. FIG. 5B shows noncovalent coupling of antibodies to the antigens that decorate the outer nanoparticle surfaces. Impurities were selectively excluded from binding to the nanoparticles and can therefore were removed by pouring off the supernatant after centrifugation. FIG. 5C shows the final synthesis step in which multi-arm NHS-PEG was mixed with the antibody-coated nanoparticles. The NHS-activated esters reacted with amine groups on the surface of the IgG antibody to form covalent amide bonds. The antigen-functionalized particles acted as node points in the network, whereas the antibody-functionalized PEG acted as a polymer bridge. Four structural aspects determine the topology of the network: (1) the number of antigens per nanoparticle, (2) the size of the nanoparticle, (3) the number of antibodies per polymer, and (4) the molecular weight of the polymer. The reactive groups on each end of the polymer form an amide bond with amine groups on the IgG antibody.

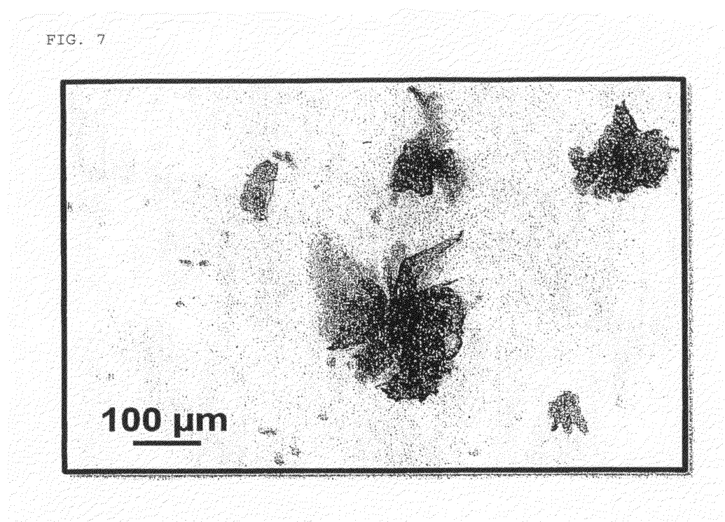

Preliminary studies showed that a 2-arm —NHS had insufficient functionality to form the hydrogel. NHS-PEG with 4 or 8 reactive groups per molecule did form a nanocomposite hydrogel. FIG. 6 shows NHS-PEG coupled to antibody. Experimentally we found that 2-arm NHS-PEG is not sufficient to form a solid hydrogel, likely due to hairpin reactions, whereby both arms of the polymer react with the same antibody. 4-arm and 8-arm NHS-PEG were sufficient to form solid hydrogels, because the many reactive groups on each polymer could tolerate one or more bonds that did not bridge between antibodies on the neighboring nanoparticles. FIG. 7 is a micrograph of an aggregated nanocomposite hydrogel formed from prostaglandin E2 functionalized magnetite nanoparticles crosslinked with anti-prostaglandin E2 antibody functionalized 4-arm PEG.

### Associative Macromolecular Assembly

The polymer that forms the basis for this technology is a macromolecular assembly comprised of antibody-functionalized polymers and antigen-functionalized polymers. When mixed together, the antigens bind to the antibodies, effectively forming a crosslinked hydrogel network (FIGS. 2, 5 and 6). Unlike normal crosslinks, these break and re-form continuously, but they spend the vast majority of their time in the bound state. This reversibility is exploited by blocking the antibodies during those rare events when the receptor/ligand complex is open. Free antigen in solution blocks the antibody, preventing the crosslink from rejoining. This phenomenon is routinely referred to as competitive binding.

The system of the invention includes a nanocomposite hydrogel consisting of two types of monomers:
1. nanoparticles functionalized with a fixed number of ligands per particle fₗ,
2. branched polymers with antibody on each end for a total of fᵣ receptor sites. f_{avg} for our system is simply (fₗ+fᵣ)/2.

fᵣ typically ranges from 2 to 8, and fₗ ranges from 2 to 1000. In an extreme example, if fᵣ=8 and fₗ=1000, then f_{ang}=504. Carothers equation then says that only 4 out of every 1000 antibodies needs to bind with a ligand in order to form a crosslinked gel. Using a large average functionality per monomer therefore compensates for relatively weak antibody/ligand bonds.

Also included in the invention are molecules having more than one antibody/antigen association. FIG. 8 (A-D) depict embodiments of the invention in which two, and exactly two, antigens are required to trigger dissolution of the polymer. Requiring a unique pair of antigens reduces the probability of false positives. The schematics also illustrate one design that results in a cascading reaction once antigens are exposed to the nanocomposite hydrogel. Cascading reactions can improve sensitivity by reducing the concentration of antigen needed to trigger dissolution. FIG. 8A illustrates the protective gel with large avidity and ligands 1 and 2 and antibodies 1 and 2 all linked together with antigen 1. FIG. 8B shows the initiation of the cascade leading to the gel breakup, once antigen 2 is introduced to the nanocomposite gel. FIGS. 8C and 8D demonstrate the continuing cascading dissociation of the gel.

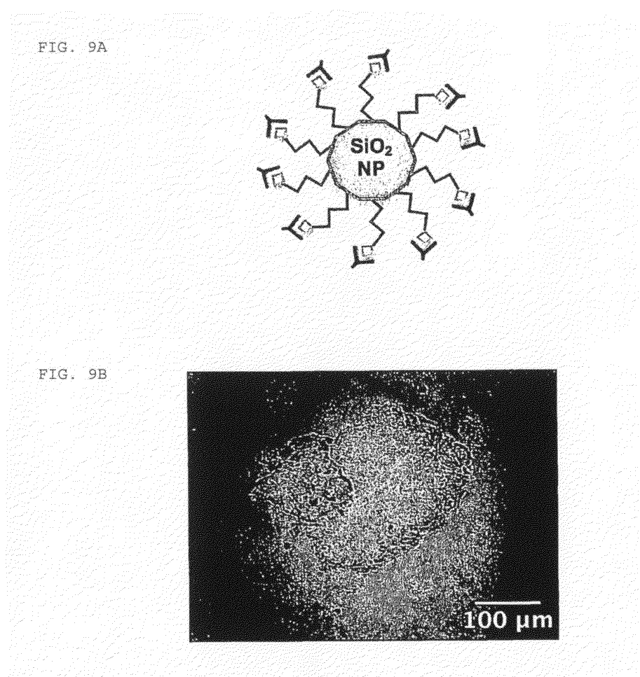

SiO₄ nanoparticles functionalized with antigens and various ligands have also been generated (FIGS. 9A and 9B). FIG. 9A depicts a silica nanoparticle embodiment of the invention. FIG. 9B shows a micrograph of a polymer complex using the SiO₂ molecule of FIG. 9A.

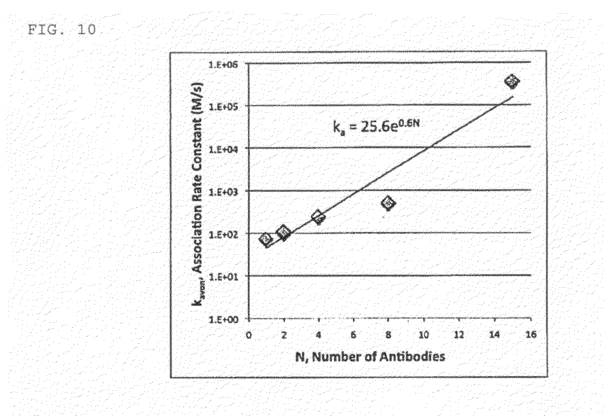

FIG. 10 is a graph depicting the increase in association rate constant that occurs when the number of antibodies per polymer (fᵣ) is increased. FIG. 10 displays surface Plasmon resonance data for multi-arm polymers functionalized with a prostaglandin E2 antibody. The increase in association rate is expected based upon the increase in avidity that occurs with each additional antibody. As was shown previously

$${k}_{avon} = {\prod }_{i}^{N} {k}_{ai}$$

The data shows that kₐᵥₒₙ had a power law relationship with the number of antibodies per polymer (N), in line with the equation above.

Having now fully described this invention, it will be understood to those of ordinary skill in the art that the same can be performed within a wide and equivalent range of conditions, formulations, and other parameters without affecting the scope of the invention or any embodiment thereof. All patents and publications cited herein are incorporated by reference in their entirety.

We claim:

1. An associative polymer complex, capable of being triggered to spontaneously release a therapeutic agent in response to a physiological event comprising,
a. a water soluble polymer backbone,
b. a molecular binding pair comprising a first binding moiety and a second binding moiety, each of the first and second binding moieties covalently attached to the water soluble polymer backbone such that the binding of the first and second binding moieties specifically to each other, cross-links the water soluble polymer backbone to form the associative polymer complex, wherein the binding strength between the first and second binding moieties is weaker than the binding of a target biomolecule specific to the first binding moiety,
c. a therapeutic agent associated with the water soluble backbone in a manner selected from the group consisting of: the agent encapsulated within the associative polymer complex, the therapeutic agent reversibly attached to the soluble polymer chain, or the therapeutic agent irreversibly attached to the soluble polymer chain, and
d. wherein upon at a predetermined concentration of the target biomolecule specific to the first binding moiety will cause the molecular binding pair to dissociate and the associative polymer to irreversibly dissolve, thus releasing the therapeutic agent, and
e. wherein the binding of the target biomolecule occurs spontaneously in response to a physiological event with no outside intervention.

2. The associative polymer complex of claim 1 further comprising a second molecular binding pair, which specifically binds a second target biomolecule, said second molecular binding pair comprising a third binding moiety and a fourth binding moiety covalently attached to the water soluble polymer backbone and wherein the binding strength between the third and fourth binding moieties is weaker than the binding of the second target biomolecule specific to the third binding moiety.

3. The associative polymer of claim 2, further comprising more than two molecular binding pairs which specifically bind to target biomolecules distinct from the first and second target biomolecules.

4. The associative polymer of claim 1, further comprising a nanoparticle coated with functional groups such that when any bond between molecular binding pairs is broken, the nanoparticle is released and the functional groups attached to the nanoparticle will attack additional bonds of any remaining unbroken molecular binding pairs.

5. The associative polymer of claim 1, wherein the molecular binding pair comprises an antibody as said first binding moiety and an antigen specific to the antibody of the first binding moiety.

6. The associative polymer of claim 1, wherein the molecular binding pair comprises two complementary strands of nucleic acids.

7. The associative polymer of claim 1, wherein the molecular binding pair comprises protein or nucleic acid aptamers.

8. The associative polymer of claim 1, further comprising a mixture of multiple binding pairs selected from the group consisting of, antibody-antigen pairs, single chain antibody-antigen pairs, nucleic acid complementary strands, protein or nucleic acid aptamer pairs, MIPS binding pairs, and receptor-ligand pairs.

9. The associative polymer of claim 4, further comprising a mixture of multiple binding pairs selected from the group consisting of, antibody-antigen pairs, single chain antibody-antigen pairs, nucleic acid complementary strands, protein or nucleic acid aptamer pairs, MIPS binding pairs, and receptor-ligand pairs.

10. An associative polymer complex, capable of being triggered to spontaneously release a thrombolytic drug in response to a thrombosis comprising,
a. a water soluble polymer backbone,
b. a molecular binding pair comprising a first binding moiety and a second binding moiety, each of the first and second binding moieties covalently attached to the water soluble polymer backbone such that the binding of the first and second binding moieties specifically to each other, cross-links the water soluble polymer backbone to form the associative polymer complex, wherein the binding strength between the first and second binding moieties is weaker than the binding of a target biomolecule specific to the first binding moiety,
c. a thrombolytic drug associated with the water soluble backbone in a manner selected from the group consisting of: the agent encapsulated within the associative polymer complex, the thrombolytic drug reversibly attached to the soluble polymer chain, or the thrombolytic drug irreversibly attached to the soluble polymer chain, and
d. wherein upon at a predetermined concentration of an eicosinoid specific to the first binding moiety will cause the molecular binding pair to dissociate and the associative polymer to irreversibly dissolve, thus releasing the thrombolytic drug, and
e. wherein the binding of the eicosinoid occurs spontaneously in response to a thrombosis event with no outside intervention.

11. The associative polymer complex of claim 10 further comprising a second molecular binding pair, which specifically binds a second target biomolecule, said second molecular binding pair comprising a third binding moiety and a fourth binding moiety covalently attached to the water soluble polymer backbone and wherein the binding strength between the third and fourth binding moieties is weaker than the binding of the second target biomolecule specific to the third binding moiety.

12. The associative polymer of claim 11, further comprising more than two molecular binding pairs which specifically bind to target biomolecules distinct from the first and second target biomolecules.

13. The associative polymer of claim 10, further comprising a nanoparticle coated with functional groups such that when any bond between molecular binding pairs is broken, the nanoparticle is released and the functional groups attached to the nanoparticle will attack additional bonds of any remaining unbroken molecular binding pairs.

14. The associative polymer of claim 10, wherein the molecular binding pair comprises an antibody as said first binding moiety and an antigen specific to the antibody of the first binding moiety.

15. The associative polymer of claim 10, wherein the molecular binding pair comprises two complementary strands of nucleic acids.

16. The associative polymer of claim 10, wherein the molecular binding pair comprises protein or nucleic acid aptamers.

17. The associative polymer of claim 10, further comprising a mixture of multiple binding pairs selected from the group consisting of, antibody-antigen pairs, single chain antibody-antigen pairs, nucleic acid complementary strands, protein or nucleic acid aptamer pairs, MIPS binding pairs, and receptor-ligand pairs.

18. The associative polymer of claim 13, further comprising a mixture of multiple binding pairs selected from the group consisting of, antibody-antigen pairs, single chain antibody-antigen pairs, nucleic acid complementary strands, protein or nucleic acid aptamer pairs, MIPS binding pairs, and receptor-ligand pairs.

19. A method of administering a therapeutic agent comprising the steps of:
a. administering to a subject in need thereof, an associative polymer complex, capable of being triggered to spontaneously release a therapeutic agent in response to a physiological event comprising,
b. a water soluble polymer backbone,
c. a molecular binding pair comprising a first binding moiety and a second binding moiety, each of the first and second binding moieties covalently attached to the water soluble polymer backbone such that the binding of the first and second binding moieties specifically to each other, cross-links the water soluble polymer backbone to form the associative polymer complex, wherein the binding strength between the first and second binding moieties is weaker than the binding of a target biomolecule specific to the first binding moiety,
d. a therapeutic agent associated with the water soluble backbone in a manner selected from the group consisting of: the agent encapsulated within the associative polymer complex, the therapeutic agent reversibly attached to the soluble polymer chain, or the therapeutic agent irreversibly attached to the soluble polymer chain, and
e. wherein upon at a predetermined concentration of the target biomolecule specific to the first binding moiety will cause the molecular binding pair to dissociate and the associative polymer to irreversibly dissolve, thus releasing the therapeutic agent, and E wherein the binding of the target biomolecule occurs spontaneously in response to a physiological event with no outside intervention, and
g. wherein the administering step comprises a method selected from the group consisting of: surgical implantation, subcutaneous injection, intramuscular injection and ingestion.

20. The method of claim 19,
a. wherein the physiological event is selected from the group consisting of high glucose levels, intravascular clot formation, atherosclerotic plague, allergic reaction, presence of toxic chemicals, presence of infectious agents, and pain, and
b. wherein the therapeutic agent that is released provides therapeutic treatment for the physiological event.

21. The method of claim 19 wherein the associative polymer complex further comprises a second molecular binding pair, which specifically binds a second target biomolecule, said second molecular binding pair comprising a third binding moiety and a fourth binding moiety covalently attached to the water soluble polymer backbone and wherein the binding strength between the third and fourth binding moieties is weaker than the binding of the second target biomolecule specific to the third binding moiety.

22. The method of claim 21 wherein the associative polymer complex further comprises more than two molecular binding pairs which specifically bind to target biomolecules distinct from the first and second target biomolecules.

23. The method of claim 19 wherein the associative polymer complex further comprises a nanoparticle coated with functional groups such that when any bond between molecular binding pairs is broken, the nanoparticle is released and the functional groups attached to the nanoparticle will attack additional bonds of any remaining unbroken molecular binding pairs.

24. The method of claim 19, wherein the molecular binding pair comprises an antibody as said first binding moiety and an antigen specific to the antibody of the first binding moiety.

25. The method of claim 19, wherein the molecular binding pair comprises two complementary strands of nucleic acids.

26. The method of claim 19, wherein the molecular binding pair comprises protein or nucleic acid aptamers.

27. The method of claim 19, further comprising a mixture of multiple binding pairs selected from the group consisting of, antibody-antigen pairs, single chain antibody-antigen pairs, nucleic acid complementary strands, protein or nucleic acid aptamer pairs, MIPS binding pairs, and receptor-ligand pairs.

28. The method of claim 21, further comprising a mixture of multiple binding pairs selected from the group consisting of, antibody-antigen pairs, single chain antibody-antigen pairs, nucleic acid complementary strands, protein or nucleic acid aptamer pairs, MIPS binding pairs, and receptor-ligand pairs.
